(19) United States
(12) Patent Application Publication
(10) Pub. No.: US 2010/0297619 A1
(43) Pub. Date: Nov. 25, 2010

(54) GENETIC PREDICTION OF SCHIZOPHRENIA SUSCEPTIBILITY

(75) Inventors: Todd Lencz, New York, NY (US); Anil K. Malhotra, Rye Brook, NY (US); John M. Kane, Long Island City, NY (US)

Correspondence Address: AMSTER, ROTHSTEIN & EBENSTEIN LLP, 90 PARK AVENUE, NEW YORK, NY, 10016, US

(73) Assignee: The Feinstein Institute for Medical Research

(21) Appl. No.: 12/311,897

(22) PCT Filed: Oct. 17, 2007
(86) PCT No.: PCT/US07/22154

§ 371 Date: Jul. 14, 2009

Related U.S. Application Data
(60) Provisional application No. 60/852,854, filed on Oct. 19, 2006.

(51) Int. Cl. C12Q 1/68

(52) U.S. Cl. 435/6

(57) ABSTRACT

Provided are methods of determining the likelihood that a patient will be diagnosed with schizophrenia.

### CROSS REFERENCE TO RELATED APPLICATION

This application claims the benefit of U.S. Provisional Patent Application No. 60/852,854, filed Oct. 19, 2006, the content of which is hereby incorporated by reference into the subject application.

### STATEMENT OF GOVERNMENT SUPPORT

This invention was made with United States government support under grant numbers MH065580, MH074543 and MH001760 from the National Institutes of Health. Accordingly, the United States government has certain rights in this invention.

### FIELD OF INVENTION

The present invention generally relates to genetic markers for predicting disease. More specifically, the invention is directed to methods of predicting susceptibility to schizophrenia.

### BACKGROUND OF THE INVENTION

Genetic epidemiologic studies have revealed high heritability estimates (70-80%) for schizophrenia, yet identification of susceptibility genes remains challenging. As with other complex diseases, linkage studies have revealed multiple candidate regions with modest LOD scores spanning large regions of the genome (Lewis et al., 2003), while studies of individual candidate genes are inherently limited in their scope and may miss unexpected loci of strong effect. By contrast, the recent development of whole genome association (WGA) technology provides an opportunity to rapidly identify novel susceptibility genes for complex phenotypes, as demonstrated in macular degeneration (Klein et al., 2005) and obesity (Herbert et al., 2006).

Based on the above, there is a need, through the application of WGA technology, for the identification of markers that are linked to likelihood of getting schizophrenia. The present invention addresses that need.

### SUMMARY OF THE INVENTION

The invention is directed to methods of determining the likelihood that a patient will be diagnosed with schizophrenia. The methods comprise determining the patient's genotype at a selected single nucleotide polymorphism (SNP). In these methods, the selected SNP is position 401 of SEQ ID NO:1, described as GenBank single nucleotide polymorphism (SNP) rs4129148. At the SNP rs4129148 SNP location, a G at position 401 of SEQ ID NO:1 is a risk allele that increases the likelihood that the patient will be diagnosed with schizophrenia. The absence of the risk allele at the selected SNP decreases the likelihood that the patient will be diagnosed with schizophrenia.

The invention is also directed to additional methods of determining the likelihood that a patient will be diagnosed with schizophrenia. These methods comprise determining whether the patient has a higher risk genotype at a selected single nucleotide polymorphism (SNP), where the selected SNP being one of the SNPs listed in Table 1 and the higher risk genotype is indicated in Table 1. In these methods, the presence of a higher risk genotype at the selected SNP increases the likelihood that the patient will get schizophrenia, and the absence of the higher risk genotype at the selected SNP decreases the likelihood that the patient will be diagnosed with schizophrenia.

Additionally, the invention is directed to methods of screening for a compound that may affect schizophrenia. The methods comprise determining whether the compound affects expression or activity of a gene listed in Table 1. In these methods, a compound that affects expression or activity of the gene may affect schizophrenia.

### BRIEF DESCRIPTION OF THE DRAWINGS

FIG. 1 is a plot of statistical significance (-log_{10P}) values for successfully genotyped SNPs in the WGA study, arranged in order of chromosomal position (1-22, X). The dotted reference line indicates threshold for genome-wide significance (P=4.2*10⁻⁷). One SNP (rs4129148) indicated with red arrow crosses this threshold (P=3.7*10⁻⁷). The dot-dashed reference line indicates P value of next highest SNP (P=2.8*10⁻⁶), separated by approximately one order of magnitude.

### DETAILED DESCRIPTION OF THE INVENTION

Markers have been identified that correlate with occurrence of schizophrenia. The identification of these markers allows the prediction of whether a patient will be diagnosed with schizophrenia.

The invention is directed to methods of determining the likelihood that a patient will be diagnosed with schizophrenia. The methods comprise determining the patient's genotype at a selected single nucleotide polymorphism (SNP). In these methods, the selected SNP is position 401 of SEQ ID NO:1, described as GenBank single nucleotide polymorphism (SNP) rs4129148. At the SNP rs4129148 SNP location, a G at position 401 of SEQ ID NO:1 is a risk allele that increases the likelihood that the patient will be diagnosed with schizophrenia. The absence of the risk allele at the selected SNP decreases the likelihood that the patient will be diagnosed with schizophrenia.

In these methods, the determination of the likelihood of schizophrenia diagnosis can be made after only determining the patient's genotype at the selected SNP, or after determining the patient's genotype at a second selected SNP. Preferably, the second selected SNP is a SNP designated in Table 1. The evaluation of the genotype of a SNP in Table 1 preferably also involves the determination of whether the patient has the risk genotype as indicated in the Table 1 column labeled “Genotypic Split”, where the genotype(s) designated in the right bracket of that column is the risk genotype(s).

The patient's genotype can be further evaluated at a third, fourth, etc. SNP associated with schizophrenia, preferably from Table 1.

The patient's genotype at a selected SNP can be linked to other SNPs, such that the genotype of the two SNPs are in linkage disequilibrium (LD) to each other. When the two SNPs are in LD, the two SNPs do not assort independently as in Hardy-Weinberg equilibrium (Balding, 2006). Under LD, the two SNPs are linked such that the prediction of the genotype at one SNP can be more and more reliably determined as LD increases by determining the genotype at the linked SNP. Thus, the genotype at a selected SNP can be reliably determined by determining the genotype at a SNP that is at high LD with the selected SNP.

The most common measures of LD are D′ and r² (Balding, 2006). With both of these measures, LD increases as D′ and r² approach 1.0. Thus, in these methods, the genotype at the selected SNP can be determined by determining the genotype at a second SNP that is at a high level of LD with the selected SNP.

Thus, in these methods, the patient's genotype at the selected SNP can be determined by determining the genotype at a secondary SNP in linkage disequilibrium to the selected SNP, where the linkage disequilibrium measure D′ between the selected SNP and the secondary SNP is greater than 0.70. Preferably, the linkage disequilibrium measure D′ is greater than 0.80; more preferably the linkage disequilibrium measure D′ is greater than 0.90.

The patient's genotype at the selected SNP can also be determined by determining the genotype at a secondary SNP in linkage disequilibrium to the selected SNP, where the linkage disequilibrium measure r² between the selected SNP and the secondary SNP is greater than 0.50. Preferably, the linkage disequilibrium measure r² between the selected SNP and the secondary SNP is greater than 0.80; more preferably the linkage disequilibrium measure r² between the selected SNP and the secondary SNP is greater than 0.90.

The invention is also directed to additional methods of determining the likelihood that a patient will be diagnosed with schizophrenia. These methods comprise determining whether the patient has a higher risk genotype at a selected single nucleotide polymorphism (SNP), where the selected SNP is one of the SNPs listed in Table 1 and the higher risk genotype is indicated in Table 1. In these methods, the presence of a higher risk genotype at the selected SNP increases the likelihood that the patient will get schizophrenia, and the absence of the higher risk genotype at the selected SNP decreases the likelihood that the patient will be diagnosed with schizophrenia.

The selected SNP can be any of those provided in Table 1, for example any SNP between the RGS18 gene and the RGS1 gene; any SNP associated with the IL1F8 gene; any SNP associated with the LRP1B gene; any SNP associated with the ROBO2 gene; any SNP associated with the IL12A gene; any SNP associated with the SLCO4C1 gene; any SNP associated with the MET gene; any SNP associated with the MCPH1 gene; any SNP associated with the KCNMA1 gene; any SNP associated with the OR2D2 gene; any SNP associated with the ST5 gene; any SNP associated with the WWOX gene; any SNP associated with the KCNJ2 gene; any SNP associated with the PLCB 1 gene; any SNP associated with the TIAM1 gene; any SNP associated with the RUNX1 gene; any SNP associated with the MN1 gene; any SNP between the GPR116 gene and the GPR110 gene; any SNP between the MTPN gene and the CHRM2 gene; any SNP between the GRIK4 gene and the LRRC35 gene; or any SNP between the CDH8 gene and the CDH11 gene.

The patient's genotype can be further evaluated at a third, fourth, etc. SNP associated with schizophrenia, preferably from Table 1.

In these methods, the patient's genotype at the selected SNP can be determined by determining the genotype at a secondary SNP in linkage disequilibrium to the selected SNP, where the linkage disequilibrium measure D′ between the selected SNP and the secondary SNP is greater than 0.70. Preferably, the linkage disequilibrium measure D′ is greater than 0.80; more preferably the linkage disequilibrium measure D′ is greater than 0.90.

The patient's genotype at the selected SNP can also be determined by determining the genotype at a secondary SNP in linkage disequilibrium to the selected SNP, where the linkage disequilibrium measure r² between the selected SNP and the secondary SNP is greater than 0.50. Preferably, the linkage disequilibrium measure r² between the selected SNP and the secondary SNP is greater than 0.80; more preferably the linkage disequilibrium measure r² between the selected SNP and the secondary SNP is greater than 0.90.

Since the markers identified in Table 1 are associated with genes, a search for compounds that could be therapeutic for schizophrenia could be conducted by screening compounds for the ability to affect expression of the genes or activity of the gene products.

The invention is thus additionally directed to methods of screening for a compound that may affect schizophrenia. The methods comprise determining whether the compound affects expression or activity of a gene listed in Table 1. In these methods, a compound that affects expression or activity of the gene may affect schizophrenia.

It is envisioned that any type of compound can be tested in these methods, including but not limited to inorganic chemicals such as metals; small organic compounds, e.g., less than 2000 or 1000 or 500 molecular weight; oligonucleotides (i.e., less than about 50 nt) such as ribozymes, aptamers and miRNA; polynucleotides such as vectors; oligopeptides; and polypeptides such as enzymes.

These screening methods can use any means known to determine whether the compound affects the expression or activity of the gene. In some aspects, the compound is contacted with a product of the gene then the activity of the gene product is measured. Here, the compound can be contacted with the product of the gene in vitro. Alternatively, the compound is contacted with a cell that expresses the product of the gene such that the compound contacts the product of the gene.

The compound can also be contacted with a cell that is capable of expressing the gene. Here, expression of the gene can be measured and compared to expression of the gene in a cell that is not contacted with the compound.

Additionally, the compound can be administered to a mammal and activity of a product of the gene can then be measured and compared to activity of the product of the gene in a mammal that is not administered the compound. Similarly, the compound can be administered to a mammal and expression of the gene is measured and compared to expression of the gene in a mammal that is not administered the compound.

These methods can measure the effect of the compound on any of the genes identified in Table 1, including the CSF2RA gene, the RGS18 gene, the RGS1 gene, the IL1F8 gene, the LRP1B gene, the ROBO2 gene, the IL12A gene, the SLCO4C1 gene, the MET gene, the MCPH1 gene, the KCNMA1 gene, the OR2D2 gene, the ST5 gene, the WWOX gene, the KCNJ2 gene, the PLCB1 gene, the TIAM1 gene, the RUNX1 gene, the MN1 gene, the GPR116 gene, the GPR110 gene, the MTPN gene, the CHRM2 gene, the GRIM gene, the LRRC35 gene, the CDH8 gene and the CDH11 gene.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### Example 1

### A Pseudoautosomal Cytokine Reteptor Gene Locus in Schizophrenia

### Example Summary

Schizophrenia is a complex disorder that affects nearly 1% of the population worldwide. Although heritability is high, few susceptibility genes have been clearly established. Reported here are results of a case-control whole genome association study examining ˜500,000 markers, which revealed a strong effect of a novel locus near the colony stimulating factor 2 receptor, alpha,low-affinity gene (CSF2RA) in the pseudoautosomal region. Sequencing of CSF2RA and its neighbor, the interleukin 3 receptor alpha, low affinity gene (IL3RA) in an independent case-control cohort revealed both common intronic polymorphisms and rare missense variants associated with schizophrenia. While consistent with other epidemiologic and biologic evidence, these findings raise new questions concerning pathophysiologic mechanisms in schizophrenia.

Reported here are results of a WGA study in a schizophrenia case-control cohort using a ˜500,000 SNP array and data from exonic sequencing in an independent case-control cohort.

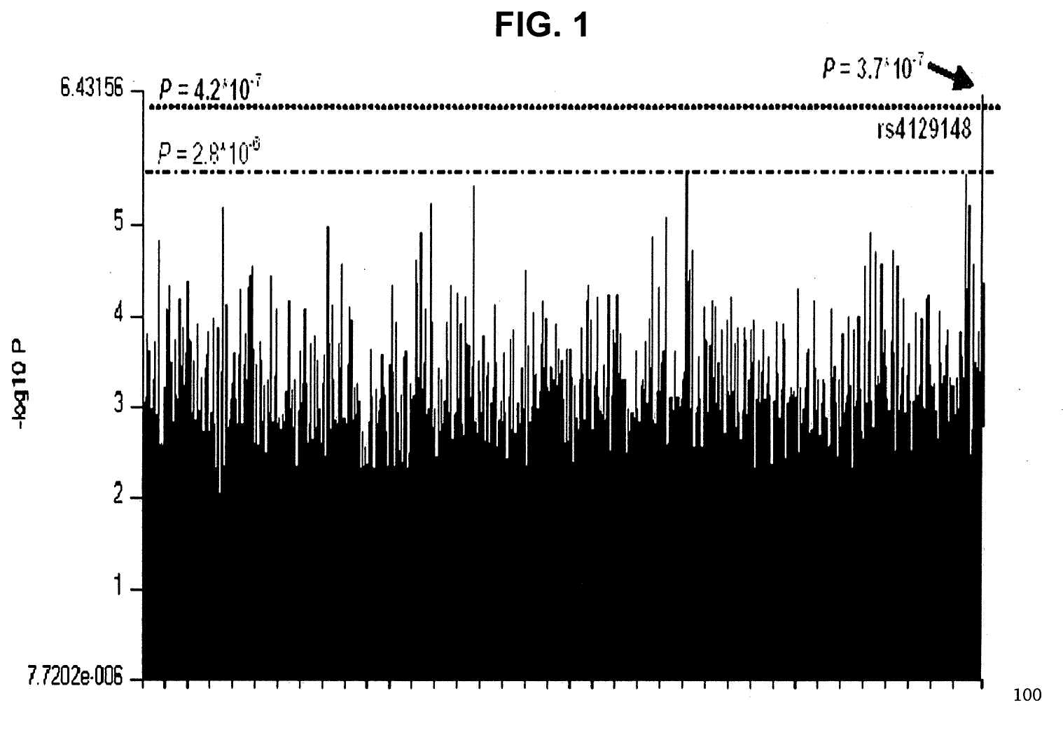

Caucasian cases (n=178) and controls (n=144) drawn from a single geographic site were assayed at 500,568 SNPs. Of these, 437,634 SNPs survived quality control procedures and were analyzed (see Materials and Methods below). Because full Bonferroni correction is overly conservative, given linkage disequilibrium across tested markers (Klein et al., 2005; Ross et al., 2001), Bayesian reasoning was utilized with conservative assumptions (Freimer and Sabatti, 2004) to establish an initial genome-wide threshold (P<4.2*10⁻⁷) for gene(s) to pursue for further examination with sequencing. As shown in FIG. 1, one SNP (rs4129148), neighboring the colony stimulating factor 2 receptor alpha, low affinity gene (CSF2RA), demonstrated an association beyond this threshold. CSF2RA is located in the pseudoautosomal region (PAR1) of the X and Y chromosomes (Xp22.32/Yp11.3). Under a dominant genetic model, homozygosity for the C allele (-strand) was significantly associated with schizophrenia; 59% of cases (105/178), but only 31% of controls (44/143, one subject not called) were C/C homozygotes (odds ratio=3.23; 95% confidence interval =2.04-5.15; population attributable risk =23.5%).

More than 1000 SNPs that showed an elevated association with schizophrenia in the WGA study were further evaluated. Of these, 175 were identified that were also associated with genes that may have a role in schizophrenia. Those 175 are identified in Table 1.

TABLE 1
SNPs with elevated association with schizophrenia
that are also associated with genes that may have a role in schizophrenia.

| | Assoc. | | | Physical | Genotypic | Risk | |
|---|---|---|---|---|---|---|---|
| SNP ID | Gene(s)^{a} | Ch^{b} | Cytoband | Position^{c} | Split^{d} | Allele | Flanking Sequence |
| rs6685520 | TNFSF4/ | 1 | q25.1 | 170009826 | {A_A,A_A}{B_B} | G | catcactcaatggaga[A/G]atatcaatgtcacatg |
| | PRDX6 | | | | | | |
| rs1281296 | GLUL/ | 1 | q25.3 | 178638368 | {A_A,A_B}{B_B} | T | ttttgtttaatgtcta[C/T]gaaccttttgaaggct |
| | CACNA1E | | | | | | |
| rs10922204 | PLA2G4A | 1 | q31.1 | 186244629 | {B_B,A_B}{A_A} | G | ggaggatattaaagac[G/T]gaactaccacaatttc |
| rs1181652 | RGS18/ | 1 | q31.2 | 188912871 | {A_A,A_B}{B_B} | G | tgtttacaggcaagcc[A/G]ctactctaaaaactac |
| | RGS1 | | | | | | |
| rs1175165 | RGS18/ | 1 | q31.2 | 188912928 | {A_A,A_B}{B_B} | T | aatcacagctaaccag[G/T]agtagaatctcacatc |
| | RGS1 | | | | | | |
| rs1175114 | RGS18/ | 1 | q31.2 | 188937134 | {B_B,A_B}{A_A} | A | gaccaactcaggctga[A/G]taaaatttcggaatca |
| | RGS1 | | | | | | |
| rs1006122 | IL1F8 | 2 | q13 | 113502073 | {A_A}{B_B,A_B} | G | gttcccgtttttaaca[C/G]tctaatgccctgcttc |
| rs2197578 | IL1F8 | 2 | q13 | 113506784 | {A_A}{B_B,A_B} | T | aagaccagtaaaggca[C/T]tctcctcaactcagca |
| rs1900287 | IL1F8 | 2 | q13 | 113513796 | {A_A}{B_B,A_B} | T | cagcctgcccacctta[C/T]gaaactitcaaaaatg |
| rs17715867 | DPP10 | 2 | q14.1 | 115096613 | {A_A}{A_B,B_B} | C | aagacatcatcagaga[A/C]aaagcaaatgtcaagt |
| rs16855067 | LRP1B | 2 | q22.1 | 142197608 | {A_A,A_B}{B_B} | T | aacaggtaaaaatgta[C/T]gcaggtaataatattt |
| rs11680286 | LRP1B | 2 | q22.2 | 142690255 | {B_B}{A_B,A_A} | A | tectaaattagaactc[A/C]attttctcctctgcac |
| rs1375613 | LRP1B | 2 | q22.2 | 142707985 | {A_A}{A_B,B_B} | T | aattggttgaagggta[A/T]atggaaactctgtatt |
| rs7577769 | LRP1B/ | 2 | q22.2 | 142976150 | {B_B,A_B}{A_A} | C | cttcaggactaaatta[C/T]gttatgcagtaaatat |
| | KYNU | | | | | | |
| rs2697148 | SLC6A1 | 3 | p25.3 | 11011580 | {A_A}{A_B,B_B} | C | agcagcagaggctata[A/C]atttttttacacgtgg |
| rs2885836 | RARB/ | 3 | p24.2 | 25170127 | {A_A,A_B}{B_B} | T | taactacagtagcgaa[C/T]tatttatcaaacaact |
| | THRB | | | | | | |
| rs1986236 | ROBO2 | 3 | p12.3 | 76084917 | {A_A}{A_B,B_B} | T | aaaaaccttcttccca[C/T]gaatgttataggagcc |
| rs9876344 | ROBO2 | 3 | p12.3 | 77388581 | {A_A}{A_B,B_B} | G | cagagcatgaaaccaa[A/G]gctctcgaggaacacc |
| rs1348213 | ROBO2 | 3 | p12.3 | 77398339 | {B_B}{A_B,A_A} | A | ttaaagtagctgagga[A/G]tagctgaacaagacct |
| rs498046 | IL12A | 3 | q25.33 | 161171920 | {B_B}{A_B,A_A} | C | aggaagctgggagtgc[C/T]gcgaatcagtgtaggg |
| rs666251 | IL12A | 3 | q25.33 | 161172102 | {A_A}{A_B,B_B} | T | acagcctaaggtgcaa[C/T]tggttgtgggaatggg |
| rs6441282 | IL12A | 3 | q25.33 | 161181779 | {A_A}{A_B,B_B} | C | ctattatgcaactatc[A/C]aaaagattgaggcaga |
| rs692890 | IL12A | 3 | q25.33 | 161182275 | {B_B,A_B}{A_A} | C | gacttttgcatttaca[C/T]agtgacattagctctt |
| rs1011927 | FXR1 | 3 | q26.33 | 182119446 | {B_B}{A_A,A_B} | A | tcaagattcagactct[A/G]aagtttgacaccacag |
| rs1805572 | FXR1 | 3 | q26.33 | 182129965 | {B_B}{A_A,A_B} | A | ttcccatacatatgca[A/G]acacagaagatttcaa |
| rs2928297 | FLJ11017 | 4 | p14 | 37280167 | {B_B}{A_B,A_A} | C | ccaagattcagggtta[C/T]cagaaaaattgtgtaa |
| rs2973226 | FLJ11017 | 4 | p14 | 37280665 | {A_A}{A_B,B_B} | G | aatgcctgacaacact[C/G]agtcagagttttaatt |
| rs10003163 | TLR10/ | 4 | p14 | 38567917 | {A_B,B_B}{B_A} | C | ttccagcgttttataa[C/T]gatgtgtcttggtatg |
| | KLF3 | | | | | | |
| rs16852350 | NSUN7 | 4 | p14 | 40416179 | {B_B}{A_B,A_A} | A | aagatgtgcttaccaa[A/T]ttctctggttagattt |
| rs17660729 | APBB2 | 4 | p14 | 41044842 | {A_A}{A_B,B_B} | G | gatcatacccaattca[C/G]aatttgaaacctgtct |
| rs6596209 | SLCO4C1 | 5 | q21.1 | 101307948 | {A_A,A_B}{B_B} | G | tatgttctttatgaca[A/G]tctaactaatgcctga |
| rs7719986 | SLCO4C1 | 5 | q21.1 | 101327048 | {A_A,A_B}{B_B} | T | aggtacagagtatgaa[G/T]aaggtctcagtttctt |
| rs6873079 | SLCO4C1 | 5 | q21.1 | 101338479 | {A_B,A_A}{B_B} | T | ggaatattgacaagac[C/T]agcaattgagacagtg |
| rs6864917 | SLCO4C1 | 5 | q21.1 | 101436806 | {A_A,A_B}{B_B} | T | cctagatatataaagc[C/T]attggcataaagctat |
| rs1381967 | SLCO4C1 | 5 | q21.1 | 101466805 | {A_A,A_B}{B_B} | T | gtagcattggcataag[C/T]agctgaaaatgcattg |
| rs12658856 | SLCO4C1 | 5 | q21.1 | 101592492 | {A_B,B_B}{A_A} | A | actgtattccactcca[A/G]atcaagtcagcctcct |
| rs10051006 | SLCO6A1 | 5 | q21.1 | 101943477 | {B_B,A_B}{A_A} | C | taaactgcacccggta[C/T]gtcaccttgtagtaag |
| rs6596061 | CSF27IL3 | 5 | q23.3 | 131484927 | {B_B}{A_A,A_B} | C | cacatgaaagctaaag[C/T]agtttttaccaaatct |
| rs2243283 | IL4 | 5 | q23.3 | 132044492 | {A_A,A_B}{B_B} | G | tgggcaggaacaaaga[C/G]gtcatcttttcctccc |
| rs7735696 | GRIA1 | 5 | q33.2 | 152916373 | {A_A}{A_B,B_B} | G | ggaaaactagagacta[A/G]gaacataaaatattgt |
| rs6580024 | GRIA1 | 5 | q33.2 | 152916614 | {B_B}{A_A,A_B} | A | cactctgtgatattca[A/T]gcaatggtgaaattgt |
| rs7753111 | BMP6 | 6 | p24.3 | 7675943 | {A_A}{A_B,B_B} | T | tatagagaatccaaca[C/T]tggtacgcaataactt |
| rs16885715 | PRL | 6 | p22.3 | 22263420 | {B_B}{A_B,A_A} | A | tgctgtctaaacaaaa[A/G]tgtataattagttggg |
| rs3130348 | AGPATI | 6 | p21.32 | 32248255 | {B_B,A_B}{A_A} | A | aactcttaaaatgatg[A/C]atatgatagtctagaa |
| rs3130284 | AGPATI | 6 | p21.32 | 32248465 | {A_B,A_A}{B_B} | G | tgagtgttctgtcaca[A/G]tcaccatatatgctgt |
| rs376877 | HLA-DPA1/ | 6 | p21.32 | 33132584 | {A_B}{A_A,B_B} | G | attagctactttgtta[C/G]atccactgtgaaattt |
| | HLA-DOA | | | | | | |
| rs6458530 | GPR116/ | 6 | p12.3 | 47003117 | {A_A}{B_B,A_B} | G | cagagacacaagatct[A/G]aattctaagtctggct |
| | GPR110 | | | | | | |
| rs4714963 | GPR116/ | 6 | p12.3 | 47020959 | {A_A}{A_B,B_B} | G | ctacctttttcttaaa[A/G]tcaaaatcacaattct |
| | GPR110 | | | | | | |
| rs1334968 | GPR116/ | 6 | p12.3 | 47022138 | {B_B}{A_B,A_A} | C | ggattatggcgattca[C/T]gtcttcacatcatcac |
| | GPR110 | | | | | | |
| rs761754 | GPR116/ | 6 | p12.3 | 47026946 | {B_B}{A_B,A_A} | C | aggcataggagcaaca[C/T]gaagcaagaagggatt |
| | GPR110 | | | | | | |
| rs4449617 | GPR116 | 6 | p12.3 | 47027846 | {B_B}{A_B,A_A} | C | gtgctttggggcccca[C/G]atttggtcctgtacct |
| | GPR110 | | | | | | |
| rs10948315 | GPR116/ | 6 | p12.3 | 47050723 | {B_B}{A_B,A_A} | C | cctcatctgataccaa[C/G]taggacagccatcatt |
| | GPR110 | | | | | | |
| rs1040604 | GPR110/ | 6 | p12.3 | 47228240 | {A_B,B_B}{A_A} | A | tggctgctatggtgaa[A/G]tggtaggttggagcta |
| | TNFRSF21 | | | | | | |
| rs9381514 | GPR110/ | 6 | p12.3 | 47232015 | {B_B,A_B}{A_A} | A | tccctgatgcatggca[A/C]ctgctggagcattctc |
| | TNFRSF21 | | | | | | |
| rs586228 | IRAK1BP1 | 6 | q14.1 | 79308383 | {B_B,A_B}{A_A} | A | aacattttgggccata[A/G]gtaataaggaagtcca |
| rs70480 | IRAK1BP1 | 6 | q14.1 | 79365324 | {A_A,A_B}{B_B} | T | tttgatgttgttcaaa[C/T]atagcacattgtttta |
| rs2236543 | HMGN3 | 6 | q14.1 | 79968908 | {B_B}{A_A,A_B} | A | cagcctctgtccaaac[A/G]caaggtgaagtacaac |
| rs1368761 | MAP3K7 | 6 | q15 | 91797168 | {B_A}{A_B,B_B} | G | aatatttttctctacc[C/G]gaatccatcagctacc |
| rs9362911 | MAP3K7 | 6 | q16.1 | 92589598 | {B_B,A_B}{A_A} | A | tttattatgtaccagg[A/G]caatattctataacat |
| rs2237717 | MET | 7 | q31.2 | 115999338 | {B_A,A_B}{B_B} | G | catgattctctccaga[A/G]cactttcattgatgaa |
| rs41735 | MET | 7 | q31.2 | 116029367 | {B_B,A_B}{A_A} | C | ttccaaaattctctcc[C/T]gctgtgtcaaagcact |
| rs41736 | MET | 7 | q31.2 | 116029719 | {B_B,A_B}{A_A} | C | ccccaccttatcctga[C/T]gtaaacacctttgata |
| rs16945 | MET | 7 | q31.2 | 116032605 | {A_A,A_B}{B_B} | T | taagttgctgattacc[C/T]ggatttttctgacgat |
| rs41746 | MET | 7 | q31.2 | 116037025 | {A_A,A_B}{B_B} | T | tttcacaaagcctttg[A/T]attcagcgtttcttgt |
| rs12706735 | GRM8 | 7 | q31.33 | 125774335 | {B_B,A_B}{A_A} | A | ttgccacagcaatgca[A/G]agaattccaaatgtct |
| rs10246817 | MTPN/ | 7 | q33 | 135514902 | {B_B,A_B}{A_A} | C | taaatgaaatttgtca[C/T]gtgccagataagataa |
| | CHRM2 | | | | | | |
| rs4732203 | MTPN/ | 7 | q33 | 135515287 | {A_B,A_A}{B_B} | G | taaaaggaacagattg[A/G]agtccagaaaaaaggc |
| | CHRM2 | | | | | | |
| rs12707304 | MTPN/ | 7 | q33 | 135567675 | {B_B,A_B}{A_A} | A | actagatttcttatac[A/G]ctgctgataggaatat |
| | CHRM2 | | | | | | |
| rs940153 | CSMD1 | 8 | p23.2 | 3836547 | {B_B}{A_A,A_B} | C | acagtaacagagatga[C/G]tacaaattagaagtgg |
| rs1978844 | CSMD1 | 8 | p23.2 | 4270039 | {A_B,B_B}{A_A} | A | cgcctaagttaggtac[A/C]aaataatgtgatagca |
| rs7824209 | MCPH1 | 8 | p23.1 | 6306668 | {A_A,A_B}{B_B} | G | tatttctccctctgaa[A/G]agatgtataactatca |
| rs2916742 | MCPH1 | 8 | p23.1 | 6306745 | {A_A,A_B}{B_B} | G | tgaatattgtcagaca[C/G]agaacccaaagaagct |
| rs2922818 | MCPH1 | 8 | p23.1 | 6306930 | {A_A,A_B}{B_B} | T | atgagattctacaact[G/T]agccacttacaaaaag |
| rs2920660 | MCPH1 | 8 | p23.1 | 6307055 | {B_B,A_B}{A_A} | A | tctatttgttccttca[A/C]atctctccaggcctcc |
| rs2916741 | MCPH1 | 8 | p23.1 | 6307171 | {B_B,A_B}{A_A} | C | tagggtgcaaaacaaa[C/T]aatagcttgcatatgc |
| rs7016737 | DLC1 | 8 | p22 | 13233118 | {A_A}{B_B,A_B} | T | tgttattttgtaagca[C/T]cacattggctactctg |
| rs10441664 | SGCZ | 8 | p22 | 14082205 | {A_B,A_A}{B_B} | T | ctgatcactagtcctt[C/T]aagcactgcaggaagt |
| rs17427734 | GFRA2 | 8 | p21.3 | 21554368 | {B_B}{A_A,A_B} | A | aaccagcagcaatgaa[A/T]taaatgagtgaatatg |
| rs4506202 | GFRA2 | 8 | p21.3 | 21642357 | {B_B,A_B}{A_A} | C | ctggatgctgttatta[C/T]ggtattctaggagtca |
| rs17186996 | KCN B2 | 8 | q13.3 | 73892651 | {A_B,A_A}{B_B} | G | tttatgctcattctca[A/G]tcgttctaaaatctaa |
| rs1227223 | CTNNA3 | 10 | q21.3 | 66248213 | {A_B,B_B}{A_A} | G | caagaaaggtgattga[G/T]ggactggcaacaagtt |
| rs2452439 | CTNNA3 | 10 | q21.3 | 66375784 | {A_B,A_B}{B_B} | T | tgcccttatttgagca[C/T]aagttgagttgaaatt |
| rs1248568 | KCNMA1 | 10 | q22.3 | 78953176 | {B_B}{A_A,A_B} | A | ttcaaatggaggtata[A/C]aaacacacagaaagac |
| rs2176283 | KCNMA1 | 10 | q22.3 | 78957134 | {B_B}{A_A,A_B} | A | atactccatgctaggg[A/C]attaactacttgttga |
| rs816828 | KCNMA1 | 10 | q22.3 | 78961874 | {B_B}{A_A,A_B} | C | cagtaacaactcaata[C/T]gaatcagaaataatct |
| rs816868 | KCNMAI | 10 | q22.3 | 78982120 | {A_A}{B_B,A_B} | G | agacccagaggatgaa[A/G]taatcttatcgaggtc |
| rs816867 | KCNMAI | 10 | q22.3 | 78982443 | {B_B}{A_B,A_A} | A | ggggtgttcacaagaa[A/G]agaagatcataaacat |
| rs6583663 | SH2D4B | 10 | q22.3 | 82556682 | {B_B}{A_B,A_A} | A | ggagaaaatgggtgca[A/C]aaatataagacttcta |
| rs9787485 | NRG3 | 10 | q23.1 | 83556666 | {A_A}{B_B,A_B} | T | ccacaagtggaaacca[C/T]cacaggaaggaaaagc |
| rs11201164 | GRID1 | 10 | q23.1 | 86422471 | {A_A,A_B}{B_B} | G | tttcggcgtacatact[C/G]acaaattgctttctta |
| m2664406 | GRID1 | 10 | q23.2 | 87348256 | {B_B,A_B}{A_A} | A | tctgcttctcctttag[A/T]acttgaaaagacaaac |
| rs7129133 | OR2D2 | 11 | p15.4 | 6880837 | {A_A,A_B}{B_B} | G | gcccttgtcagacata[C/G]atgaaggaaaagtttc |
| rs6578793 | OR2D2 | 11 | p15.4 | 6881438 | {A_A,A_B}{B_B} | T | gcaaataatgtatgag[C/T]caagttacaaaaccaa |
| rs7110568 | OR2D2 | 11 | p15.4 | 6888972 | {A_A,A_B}{B_B} | T | gaggtcctgagaatta[C/T]gtcaagagggcattcc |
| rs10839656 | OR2D3 | 11 | p15.4 | 6890346 | {B_B,A_B}{B_A} | A | agaaagaggaaaagga[A/G]tacagaaagagagatg |
| rs11041095 | OR2D3 | 11 | p15.4 | 6890377 | {A_A,A_B}{B_B} | T | ctgccttctctctaca[C/T]ctctgaattcctgact |
| rs1388535 | OR2D3 | 11 | p15.4 | 6899830 | {A_A,A_B}{B_B} | T | acttttagagctatgg[C/T]aacattctttaaaaag |
| rs7479713 | OR2D3 | 11 | p15.4 | 6900632 | {A_A,A_B}{B_B} | C | gttctagtattgaaga[A/C]taatgacaattgctct |
| rs994710 | OR2D3 | 11 | p15.4 | 6901105 | {A_A,A_B}{B_B} | T | gggacttctacacagt[C/T]aatctgaggtctgtgg |
| rs2279533 | ZNF215 | 11 | p15.4 | 6904541 | {A_A,A_B}{B_B} | T | cgcccgctgacgagaa[C/T]gatacccgtgagcacc |
| rs6484484 | ST5 | 11 | p15.4 | 8773782 | {A_B,A_A}{B_B} | G | agccaataaaataggc[A/G]agtttcctcccactcc |
| rs7944560 | ST5 | 11 | p15.4 | 8773852 | {A_B,A_A}{B_B} | T | tacaaatcatattcaa[A/T]tgtgtttccttcctgc |
| rs4929914 | ST5 | 11 | p15.4 | 8780964 | {A_B,B_B}{A_A} | C | cctaccttcftactta[C/T]taaaggctgaggcctc |
| rs4929916 | ST5 | 11 | p15.4 | 8781572 | {A_B,A_A}{B_B} | T | ctagggttcagtctcc[C/T]cttccagaaagtagaa |
| rs1151488 | MAP3K11 | 11 | q13.1 | 65128180 | {A_A}{A_B,B_B} | T | agaagtttgtcactga[C/T]ctgaactattcatgaa |
| rs10895257 | YAP1 | 11 | q22.2 | 101491854 | {B_B}{A_B,A_A} | A | gggagattgttaaaca[A/G]cttaatagcctgcttt |
| rs628269 | IL10RA/ | 11 | q23.3 | 117431945 | {B_B,A_B}{A_A} | A | gcagaggaggtggtga[A/G]ggaagaagaggctcta |
| | TMPRSS4 | | | | | | |
| rs3824978 | GRIK4 | 11 | q23.3 | 120237718 | {B_B,A_B}{A_A} | C | tcacagtgttaactcc[C/T]agaaaaactgctctca |
| rs1944516 | GRIK4/ | 11 | q23.3 | 120382996 | {B_B}{A_B,A_A} | C | tgttaaatttgagatg[C/T]atatgggctatctaag |
| | LRRC35 | | | | | | |
| rs11218108 | GRIK4/ | 11 | q23.3 | 120384126 | {A_A}{A_B,B_B} | T | ccccaggttctgtaaa[C/T]tcttgagtcttgtcct |
| | LRRC35 | | | | | | |
| rs12283211 | GRIK4/ | 11 | q23.3 | 120396033 | {B_B,A_B}{A_A} | C | aaaaggagagaatgga[C/T]gttgaactaaactgag |
| | LRRC35 | | | | | | |
| rs11111347 | IGF1/PAH | 12 | q23.2 | 101606138 | {A_B}{A_A} | A | attagcaaatacatag[A/T]agacaggttttaattc |
| rs2891403 | PTPN11/ | 12 | q24.13 | 111600292 | {A_A,A_B}{B_B} | G | atgcaaattatacctc[A/G]ataaggttgatataaa |
| | RPH3A | | | | | | |
| rs9513299 | RAP2A/ | 13 | q32.2 | 97231917 | {A_A}{B_B,A_B} | G | gtagctctctctttga[A/G]ggttgcttggcagctt |
| | RANBP5 | | | | | | |
| rs9517069 | RAP2A/ | 13 | q32.2 | 97246696 | {A_A}{B_B,A_B} | C | aagcaagaaaatatga[A/C]ccagaatgaggagaaa |
| | RANBP5 | | | | | | |
| rs1570709 | DADA | 13 | q33.2 | 104831385 | {A_A}{A_B,B_B} | G | gtttcttagatccaca[A/G]actcgacacagacttt |
| rs17294476 | HOMER2 | 15 | q25.2 | 81377930 | {A_A,A_B}{B_B} | G | ccttcaaaaatctaac[A/G]cttgaccctagggaca |
| rs8051129 | A2BP1 | 16 | p13.2 | 7222984 | {B_B,A_B}{A_A} | C | gggtgacaacaatcac[C/T]ggtgtggagagggaaa |
| rs11074939 | CIITA | 16 | p13.13 | 10919210 | {A_A}{A_B,B_B} | G | acaaccaaggtcttag[A/G]atggccaaataattta |
| rs12923340 | CDH8/ | 16 | q21 | 61448349 | {A_A}{B_B,A_B} | T | tgccctctgattcag[C/T]ccaataagtaatttag |
| | CDH11 | | | | | | |
| rs12598906 | CDH8/ | 16 | q21 | 61533104 | {B_B}{A_B,A_A} | C | acccttgatggacatt[C/T]aagtttattccatgtc |
| | CDH11 | | | | | | |
| rs30873 | CDH8/ | 16 | q21 | 61544568 | {A_A}{B_B,A_B} | G | aagcgtaaatgccact[A/G]aagaaaatttaaaaga |
| | CDH11 | | | | | | |
| rs39596 | CDH8/ | 16 | q21 | 61549040 | {A_A}{B_B,A_B} | G | tctttttctctattcc[A/G]ccatttagaattctgc |
| | CDH11 | | | | | | |
| rs43172 | CDH8/ | 16 | q21 | 61551684 | {B_B}{A_A,A_B} | C | aggtcacagttgaaca[C/T]tgccattatctgaaac |
| | CDH11 | | | | | | |
| rs30888 | CDH8/ | 16 | q21 | 61561931 | {B_B}{A_A,A_B} | A | agtttgaaggtgcatg[A/G]atttttgacattgtgc |
| | CDH11 | | | | | | |
| rs368182 | CDH9/ | 16 | q21 | 61579981 | {A_A}{B_B,A_B} | T | atgaatgaacaggtga[C/T]gaataacaagtgaata |
| | CDH11 | | | | | | |
| rs585417 | CDH8/ | 16 | q21 | 61581118 | {A_A}{A_B,B_B} | T | gaagagtaaactgtaa[C/T]agcacttggagcagtg |
| | CDH11 | | | | | | |
| rs588037 | CDH8/ | 16 | q21 | 61581641 | {A_A}{B_B,A_B} | T | aggcactttttctcaa[C/T]gtatcaataactttgc |
| | CDH11 | | | | | | |
| rs11075613 | CDH11/ | 16 | q22.1 | 64612210 | {B_B,A_B}{A_A} | C | gcaatgagtcagaaca[C/G]gtacagatgactcagc |
| | CDH5 | | | | | | |
| rs6564592 | WWOX | 16 | q23.1 | 77396857 | {B_B}{A_B,A_A} | C | ctgactctacgaaaaa[C/T]atgaaaaatacaaaca |
| rs7499973 | WWOX | 16 | q23.1 | 77399684 | {A_A}{A_B,B_B} | G | gttcctgggttgcaaa[C/G]ggatccatagtccaaa |
| rs1072247 | WWOX | 16 | q23.1 | 77416144 | {A_A}{A_B,B_B} | T | cgaggtaactcaatca[C/T]gtgggccggtctttcc |
| rs11080004 | CA10 | 17 | q22 | 47802800 | {A_A}{A_B,B_B} | T | gtggcattgtcaatta[C/T]atttgaagatgcacat |
| rs2058083 | KCNJ2 | 17 | q24.3 | 66649998 | {B_B}{A_A,A_B} | A | attttgagaagacacc[A/G]acaagcgaatatgcat |
| rs2058084 | KCNJ2 | 17 | q24.3 | 66650612 | {A_A}{B_B,A_B} | T | atgaagcatgtgttta[C/T]aaagctaaagatatgt |
| rs9915190 | KCNJ2 | 17 | q24.3 | 66654223 | {B_B}{A_A,A_B} | A | ttttcaatggtttatc[A/C]cagatgacagctgatt |
| rs723338 | KCNJ2 | 17 | q24.3 | 66657008 | {B_B}{A_B,A_A} | C | aatgtccaagcgacat[C/T]aggtgttttagtacag |
| rs8069925 | KCNJ2 | 17 | q24.3 | 66676457 | {B_B}{A_A,A_B} | G | ataactttggttccaa[G/T]attttgattttctaat |
| rs9907418 | KCNJ2 | 17 | q24.3 | 66676814 | {A_A}{B_B,A_B} | G | aaacttcgcattccca[A/G]tatgaagaagtgataa |
| rs2367263 | KCNJ2 | 17 | q24.3 | 66677883 | {B_B}{A_A,A_B} | A | attgccttggtagaaa[A/G]gagtatggtgggcacc |
| rs1859964 | KCNJ2 | 17 | q24.3 | 66678166 | {A_A}{A_B,B_B} | C | tctgccaaaggtaact[A/C]aattttatccagaact |
| rs6501447 | KCNJ2 | 17 | q24.3 | 66684693 | {B_B}{A_A,A_B} | C | gttgtcacagccagaa[C/T]attttacaacagtgtg |
| rs17178251 | KCNJ2 | 17 | q24.3 | 66688474 | {B_B}{A_A,A_B} | C | ggcagttttgatggag[C/G]ctcaagagatacagtc |
| rs17765644 | KCNJ2 | 17 | q24.3 | 66691087 | {B_B}{A_A,A_B} | C | cagcccaactaaacca[C/T]caactccaagactcac |
| rs9913988 | KCNJ2 | 17 | q24.3 | 66691653 | {B_B}{A_A,A_B} | C | cccagcacgatagcta[C/T]gttttagggatactga |
| rs758106 | KCNJ2 | 17 | q24.3 | 66692598 | {B_B}{A_A,A_B} | C | ctctcggctacccttc[C/T]caatccttgctcacca |
| rs11870732 | KCNJ2 | 17 | q24.3 | 66706836 | {B_B}{A_A,A_B} | C | gggctcatgaactgaa[C/T]agagggaatccagaaa |
| rs4793541 | KCNJ2 | 17 | q24.3 | 66739190 | {A_A}{B_B,A_B} | G | ggaagaggttcagagc[A/G]aagtggtggaaaagcc |
| rs8070461 | KCNJ2 | 17 | q24.3 | 66752467 | {B_B}{A_A,A_B} | A | gataacaatccaacac[A/C]gtgcttatggagcatg |
| rs9891216 | KCNJ2 | 17 | q24.3 | 66754527 | {A_A}{A_B,A_B} | T | tggtatgatctgaatt[C/T]aagagtcdttctggc |
| rs12052015 | BCL2/ | 18 | q21.33 | 58820417 | {A_A,A_B}{B_B} | G | tccccaaacaactgca[C/G]ctcctctcacagccaa |
| | PHLPP | | | | | | |
| N/A | CASP14 | 19 | p13.12 | 15015126 | {A_B,B_B}{A_A} | A | aatcctgggctaatcc[A/G]aggctagttcttccca |
| rs4815994 | BMP2/ | 20 | p12.3 | 7220127 | {B_B,A_B}{A_A} | A | ttaccagcctctagga[A/G]atgattaatcaagaag |
| | HAO1 | | | | | | |
| rs6118338 | PLCB1 | 20 | p12.3 | 8760751 | {A_B,B_B}{A_A} | C | ttttaatgccagaatc[C/T]gacttatgaattcact |
| rs6516415 | PLCB1 | 20 | p12.3 | 8794006 | {A_B,B_B}{A_A} | G | aatgtttatgccataa[G/T]gatggcctcttaaatt |
| rs6118364 | PLCB1 | 20 | p12.3 | 8798286 | {A_B,B_B}{A_A} | A | atggggaaatgcaaca[A/G]taataccaaataacag |
| rs2833336 | TIAM1 | 21 | q22.11 | 31503585 | {A_A,A_B}{B_B} | G | aaaccaggttcacttt[A/G]aaaaagatcaaggcat |
| rs2257060 | TIAM1 | 21 | q22.11 | 31630052 | {B_B,A_B}{A_A} | A | gataacacacagttca[A/T]ctccagggtcactgcc |
| rs2833378 | TIAM1 | 21 | q22.11 | 31630312 | {B_B,A_B}{A_A} | A | aagatgtccaagacag[A/C]ataagtgaagacagac |
| rs2833379 | TIAM1 | 21 | q22.11 | 31630474 | {A_A,A_B}{B_B} | T | tctgttgaactacatc[C/T]gaagtactgggggaga |
| rs2252002 | TIAM1 | 21 | q22.11 | 31632892 | {A_B,A_A}{B_B} | T | acgttgcacggcgacc[G/T]cttgatccattctgtg |
| rs2833384 | TIAM1 | 21 | q22.11 | 31642320 | {A_B,A_A}{B_B} | G | tgaaaggaattttaaa[A/G]cccagctattgtagat |
| rs2242888 | RUNX1 | 21 | q22.12 | 35309698 | {B_B}{A_B,A_A} | C | taatgagatacaatga[C/T]gccagtggtggaagag |
| rs928284 | RUNX1 | 21 | q22.12 | 35356044 | {B_B,A_B}{A_A} | A | aggagattttagccca[A/G]agaggttatgatacag |
| rs2834755 | RUNX1 | 21 | q22.12 | 35385708 | {B_B,A_B}{A_A} | G | atacaaaattgggagg[G/T]aactgctatgaggtaa |
| rs2032148 | RUNX1 | 21 | q22.12 | 36293953 | {A_A}{A_B,B_B} | T | gcttgttaaaactgca[C/T]catctaggtcctcatc |
| rs2835226 | RUNX1 | 21 | q22.12 | 36294554 | {A_A}{A_B,B_B} | T | agatgaggctttcaga[C/T]atagagcctccttaaa |
| rs2835261 | RUNX1 | 21 | q22.12 | 36349777 | {B_B}{A_B,A_A} | A | ttttgactgaagccca[A/G]aaatgttacctcctat |
| rs5762162 | MN1 | 22 | q12.1 | 26186476 | {A_B,A_A}{B_B} | G | cagcgaaagtccaaat[A/G]atgcagacaaaagtgg |
| rs7292844 | MN1 | 22 | q12.1 | 26218051 | {A_B,A_A}{B_B} | T | ccaccacaactggcaa[C/T]gttttatttttttgt |
| rs2024682 | MN1 | 22 | q12.1 | 26230380 | {B_B,A_B}{A_A} | A | tgaatctgacatagtc[A/C]cacctgcagagaactc |
| rs9614284 | SULT4A1/ | 22 | q13.2 | 42588872 | {B_B,A_B}{A_A} | A | gcacgctgggtgacca[A/C]aactcccgacgcccct |
| | PNPLA5 | | | | | | |
| rs4129148 | CSF2RA/ | X | p22.33 | 960180 | {B_B,A_B}{A_A} | C | gagagaaattcagacc[C/G]agttgcattggccatt |
| | IL3RA | | | | | | |
| rs5958109 | GRIA3 | X | q25 | 121672730 | {A_B,B_B}{A_A} | C | tattcactttactgta[C/G]cagtctggaaccaaat |
| rs5987140 | ABCD1 | X | q28 | 152521102 | {A_A,A_B}{B_B} | G | tgtacatggaaaagga[A/G]gaattgtatagtatgt |

^{a}Where only one gene designated, the SNP is in, or within 500,000 BP of the designated gene. Where two genes are designated, the SNP is between the two genes and less than 500,000 by away from both genes.
^{b}Chromosome number
^{c}NCBI B35 Assembly
^{d}Right bracket contains higher risk genotype(s); A/B alleles refer to first/second allele recorded in the flanking sequence entry.

These data from strongly suggest the presence of a novel schizophrenia susceptibility locus in PAR1. While there is some prior cytogenetic support for such a locus (Ross et al., 2001), linkage studies have been equivocal, and CSF2RA has not been previously implicated in schizophrenia.

A role for cytokine-related genes in mediating schizophrenia risk is consistent with several other lines of evidence: increased risk subsequent to prenatal viral exposure; altered rates of autoimmune disorders in patients with schizophrenia and their relatives; and monozygotic twin discordance (Hanson et al., 2005). Moreover, abnormalities of colony stimulating factor 2 (CSF2), interleukin-3, and their receptors have been associated with lymphoma and leukemia, the incidence of which is reduced in relatives of patients with schizophrenia (Daloton et al., 2004; Lictermann et al., 2001). Finally, while CSF2 is most commonly associated with proliferation and differentiation of granulocytes and macrophages, there is accumulating evidence of its critical role in the central nervous system repair via expression of brain derived neurotrophic factor (Bouhy et al., 2006), a trophic factor that has been associated with psychiatric illness. This is consistent with broader evidence of a role for cytokines in neuroprotection and behavioral phenotypes (Licinio and Wong, 1997). Further studies are needed to determine whether the association of this cytokine receptor genetic locus with schizophrenia is mediated by immune response to infectious agents, autoimmune or inflammatory processes, trophic factors, or a combination of these mechanisms.

### Materials and Methods

Participants. For the WGA study, patients with schizophrenia (n=158), schizoaffective disorder (n=13), or schizophreniform disorder (n=7) were recruited from the inpatient and outpatient clinical services of The Zucker Hillside Hospital, a division of the North Shore-Long Island Jewish Health System. After providing written informed consent, the Structured Clinical Interview for DSM-IV Axis I disorders (SCID, version 2.0) was administered by trained raters. Information obtained from the SCID was supplemented by a review of medical records and interviews with family informants when possible; all diagnostic information was compiled into a narrative case summary and presented to a consensus diagnostic committee, consisting of a minimum of three senior faculty. Healthy controls (n=144) were recruited by use of local newspaper advertisements, flyers, and community Internet resources and underwent initial telephone screening to assess eligibility criteria. The nonpatient SCID (SCID-NP) was administered to subjects who met eligibility criteria, to rule out the presence of an Axis I psychiatric disorder; a urine toxicology screen for drug use and an assessment of the subject's family history of psychiatric disorders were also performed. Exclusion criteria included (current or past) Axis I psychiatric disorder, psychotropic drug treatment, substance abuse, a first-degree family member with an Axis I psychiatric disorder, or the inability to provide written informed consent. Patients (65 female/113 male) and controls (63F/81M) did not significantly differ in sex distribution (P >0.05). All self-identified as Caucasian, non-Hispanic. Population structure was tested by examination of 210 ancestry informative markers (AIMs). AIMs included all SNPs on the array that passed initial quality control procedures and demonstrated a frequency difference of >0.5 in comparisons between Caucasian individuals and Asians or African-Americans in data made publicly available by Shriver and colleagues (Shriver et al., 2003; http://146.186.95.23/biolab/voyage/psa.html). Only 4 AIMs (2%) demonstrated allelic frequency differences between cases and controls at P<0.05, a result not different from chance.

The sample for the sequencing study was drawn from a larger sample of 85 schizophrenia patients (74 Caucasian, 8 Native American, and 3 African American by self report) and 66 healthy subjects of various self-reported ethnicities. All 151 subjects were genotyped at 67 AIMs and analyzed using the STRUCTURE program (Pritchard et al., 2000). The 71 Caucasian cases (28F/43M) and 31 controls (18F/13M) who were at least 90% Caucasian were used in the case-control analysis; groups did not significantly differ on sex distribution (P>0.05). All cases were diagnosed with schizophrenia and were drawn from a nationwide population of clozapine-treated patients in the United States as part of a larger study on clozapine-induced agranulocytosis; 26 patients (12F/14M) in the sample had developed agranulocytosis while undergoing treatment with clozapine. Importantly, significant case-control differences in CSF2RA and IL3RA genotypes were not substantially derived from agranulocytosis cases, as will be described in more detail below.

WGA Genotyping and Analysis. Genomic DNA extracted from whole blood was hybridized to two chips containing ˜0.262,000 and ˜0.238,000 SNPs as per manufacturer's specifications (Affymetrix, Santa Clara, Calif.; S3). Genotype calls were obtained using the Bayesian Robust Linear Model with Mahalanobis distance classifier (BRLMM) algorithm thresholded at 0.5 applied to batches of 100 samples. Quality control procedures followed several steps. First, samples that obtained mean call rates <90% across both chips (or <85% for a single chip) were rejected. Mean call rate of remaining samples (total n=322) was 97%. Twenty-two of these cases were successfully repeated, and concordance of the two calls (reliability) for each SNP was evaluated. SNPs with >1 discrepancy were excluded from further analyses. Concordance across the remaining 454,699 SNPs exceeded 99%. An additional 1526 SNPs with call rates <0.85 across all valid cases were excluded, as were 13,662 SNPs not in Hardy-Weinberg equilibrium (P<0.001) in the control sample, yielding 439,511 SNPs available for analysis. Likelihood ratios (df=1) were analyzed for the best possible genotypic split (e.g., recessive or dominant models) for each SNP, with the constraint that a minimum of 10 subjects populate each split group (thereby excluding monomorphic and very rare SNPs), yielding 362,188 splits plotted in FIG. 1. All statistical analyses described above were conducted using HelixTree 5.0.2 software (GoldenHelix, Bozeman, Mont.).

Because strict Bonferroni correction is considered overly conservative, a Bayesian formula was applied to obtain >0.95 posterior probability of a correct inference of association to a particular gene; this approach was advocated by Freimer and Sabatti (2004), and modified to take into account recent estimates of the total number of genes in the human genome to approximate ˜20,000, resulting in a P value threshold of ˜4.2×10⁻⁷. This gene-wise approach is particularly applicable to the current study design, in which the WGA analysis is conducted with the assumption that significant marker(s) are not themselves functional variants but rather indicate neighboring genetic loci to be examined in greater detail. It could be argued that this threshold is also overly conservative, in that it assumes a single genetic locus and does not take into account prior information (e.g. linkage data) that may provide greater likelihood in particular regions of the genome. However, such information is difficult to quantify (S4) and therefore was not included in deriving the threshold. Finally, it should be noted that empirical testing of all 362,188 P values using the Q-value program (Storey and Tibshirani, 2003) yields a false discovery rate g<0.05 for the threshold applied in the present study.

Sequencing and analysis. The following regions were sequenced: CSF2RA, exons 3, 7, 8, 9, 12 and an upstream region; IL3RA, exons 1-3,5-8, 10 and 12 (reference GenBank mRNA accession numbers NM 006140.3 and NM 002183.2, respectively). Fragments were amplified using primers designed by routine methods. Primers were tailed with M13F for forward primers and with M13R for reverse primers. The sequencing reactions were carried out in both directions using M13F and M13R primers using BigDye™ Terminator Cycle Sequencing, and electrophoresis was run on the ABI Prism 3700 DNA Analyzer, according to standard procedures. Fragments were blasted against the target sequence and polymorphisms were scored for fragments with an average phred score >30. As a control, Mendelian inheritance of each polymorphism was assessed in one

Caucasian family (4 grandparents, 2 parents, 4 children) and one African American family (2 parents, 5 children) and population specific Hardy-Weinberg equilibrium was determined for 79 unrelated individuals.

### REFERENCES

D. J. Balding, Nature Reviews I Genetics 7, 781 (2006).
J. C. Barrett, B. Fry, J. Mailer, M. J. Daly, Bioinformatics 21, 263 (2005).
D. Bouhy et al., FASEB J. 20, 1239 (2006).
S. O. Dalton, T. M. Laursen, L. Mellemkjmr, C. Johansen, P. B. Mortensen, Am. J. Psychiatry, 161, 903 (2004).
N. Freimer, C. Sabatti, Nature Genet. 36, 1045 (2004). D. R. Hanson, I. I. Gottesman, BMC Med. Genet. 6, 7 (2005).
A. Herbert et al., Science 312, 279 (2006).
G. C. Kennedy, et al., Nature Biotechnol. 21, 1233 (2003).
R. J. Klein et al., Science 308, 385 (2005).
C. M. Lewis et al., Am. J. Hum. Gen. 73, 34 (2003).
D. Lichtermann, J. Ekelund, E. Pukkula, A. Tanskanen, J. LOnnqvist, Arch. Gen. Psychiatry, 58, 573 (2001).
J. Licinio, M-L. Wong, J. Clin. Invest. 100, 2941 (1997).
J. K. Pritchard, M. Stephens, P. Donnelly, Genetics 155, 945 (2000).
N. L. Ross et al., J. Med. Genet. 38, 710 (2001).
M. D. Shriver et al., Hum. Genet. 112, 387 (2003).
J. D. Storey, R. Tibshirani, Proc. Natl. Acad. Sci. U.S.A. 100, 9440 (2003).

SEQ ID NO: 1
S = G or C
rs4129148

| | |
|---|---|
| 1 | CTTTGGCAGG GAGGTTTTTA TTAAAATGAT |
| | TAAGGACCGA GGTGGTGATC CCGGGAAAAG |
| 61 | TTGTTGAAAA ATGAAAGTTT GGGCCTCTGA |
| | GTTAATGGTT TAATTATGCG TCTCAGGATT |
| 121 | TGCAAAGAGC ACAGTCATCT TATGTGTGGG |
| | GCGGATTCTG ACTTCCCGTC CAGCCTTTCA |
| 181 | TTTCTAGAGA CAATCGGCCA ATTTATACTT |
| | AGGGGAAAAT GAAGGACGAG CTGAAAGAAT |
| 241 | GGCAGAAAGT CCATCTTCCA AGCTCTTCCT |
| | GGAAATACAG AAAAGAAAGG AGCGTCCTCA |
| 301 | AAAAACACCA GCCCACACGT TGACGTCGGC |
| | ACCTCCTGCA AGATGAGCCG GGCACATTTA |
| 361 | TTAAGCAGCC GTTTGGCTGG AAAAAATGGC |
| | CAATGCAACT |
| 401 | S |
| 402 | GGTCTGAATT TCTCTCCTGG GTGTCCAAGG |
| | ATCAAGGCAG GTAATGAGGT TGGCAGAATT |
| | ATGAATCGCA ATCAAATTTT TTCTATGACC |
| | TGATAGAAAC GCCAACTCAC TGTATGAGTC |
| | TGTTGTCAAA GTCTAACTTC CAAAACGCAA |
| | AGCACGCTCC CGATCATAAA CACAGAGATA |
| | GCAGTATTGA AGAATTGTCC CCCCGGCTCC |
| | AGCCCCGGAT TTCTCTAGAG GGGCTAATGG |
| | AGGCCACGAC ATTTGTGGCA TAAAAAGGTT |
| | TGCGATGAAC AGTCTTGCCT GGGGTGCAGG |
| | AAGGTGAGGA GCTGTGTGTG GCTTTTGCGT |
| | TTTCCTTCAC GATGGAATGC GAGTCTTTGA |
| | TGCCGGCTAG ATCCACGTTT TATGAGGCGG |
| | AATTTTTAAT |

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

1. A method of determining the likelihood that a patient will be diagnosed with schizophrenia, the method comprising
determining the patient's genotype at a selected single nucleotide polymorphism (SNP),
wherein the selected SNP is position 401 of SEQ ID NO:1, described as GenBank single nucleotide polymorphism (SNP) rs4129148,
wherein a G at position 401 of SEQ ID NO:1 is a risk allele that increases the likelihood that the patient will be diagnosed with schizophrenia, and the absence of the risk allele at the selected SNP decreases the likelihood that the patient will be diagnosed with schizophrenia.

2. The method of claim 1, further comprising determining the patient's genotype at a second selected SNP.

3. The method of claim 2, wherein the second selected SNP is a SNP designated in Table 1.

4. The method of claim 1, wherein the patient's genotype at the selected SNP is determined by determining the genotype at a secondary SNP in linkage disequilibrium to the selected SNP, wherein the linkage disequilibrium measure D′ between the selected SNP and the secondary SNP is greater than 0.70.

5. The method of claim 4, wherein the linkage disequilibrium measure D′ is greater than 0.80.

6. The method of claim 4, wherein the linkage disequilibrium measure D′ is greater than 0.90.

7. The method of claim 1, wherein the patient's genotype at the selected SNP is determined by determining the genotype at a secondary SNP in linkage disequilibrium to the selected SNP, wherein the linkage disequilibrium measure r² between the selected SNP and the secondary SNP is greater than 0.50.

8. The method of claim 4, wherein the linkage disequilibrium measure r² between the selected SNP and the secondary SNP is greater than 0.80.

9. The method of claim 4, wherein the linkage disequilibrium measure r² between the selected SNP and the secondary SNP is greater than 0.90.

10. A method of determining the likelihood that a patient will be diagnosed with schizophrenia, the method comprising determining whether the patient has a higher risk genotype at a selected single nucleotide polymorphism (SNP), the selected SNP being one of the SNPs listed in Table 1 and the higher risk genotype is indicated in Table 1, wherein the presence of a higher risk genotype at the selected SNP increases the likelihood that the patient will get schizophrenia, and the absence of the higher risk genotype at the selected SNP decreases the likelihood that the patient will be diagnosed with schizophrenia.

11. The method of claim 10, wherein the selected SNP is between the RGS18 gene and the RGS1 gene.

12. The method of claim 10, wherein the selected SNP is associated with the IL1F8 gene, the LRP1B gene, the ROBO2 gene, the IL12A gene, the SLCO4C1 gene, the MET gene, the MCPH1 gene, the KCNMA1 gene, the OR2D2 gene, the ST5 gene, the WWOX gene, the KCNJ2 gene, the PLCB1 gene, the TIAM1 gene, the RUNX1 gene, or the MN1 gene.

13-27. (canceled)

28. The method of claim 10, wherein the selected SNP is between the GPR116 gene and the GPR110 gene.

29. The method of claim 10, wherein the selected SNP is between the MTPN gene and the CHRM2 gene.

30. The method of claim 10, wherein the selected SNP is between the GRIK4 gene and the LRRC35 gene.

31. The method of claim 10, wherein the selected SNP is between the CDH8 gene and the CDH11 gene.

32. The method of claim 10, further comprising determining the patient's genotype at a second selected SNP.

33. The method of claim 10, wherein the patient's genotype at the selected SNP is determined by determining the genotype at a secondary SNP in linkage disequilibrium to the selected SNP, wherein the linkage disequilibrium measure D′ between the selected SNP and the secondary SNP is greater than about 0.70.

34. The method of claim 33, wherein the linkage disequilibrium measure D′ is greater than about 0.80.

35. The method of claim 33, wherein the linkage disequilibrium measure D′ is greater than about 0.90.

36. The method of claim 10, wherein the patient's genotype at the selected SNP is determined by determining the genotype at a secondary SNP in linkage disequilibrium to the selected SNP, wherein the linkage disequilibrium measure r² between the selected SNP and the secondary SNP is greater than about 0.50.

37. The method of claim 36, wherein the linkage disequilibrium measure r² between the selected SNP and the secondary SNP is greater than about 0.80.

38. The method of claim 36, wherein the linkage disequilibrium measure r² between the selected SNP and the secondary SNP is greater than about 0.90.

39. A method of screening for a compound that may affect schizophrenia, the method comprising determining whether the compound affects expression or activity of a gene listed in Table 1, wherein a compound that affects expression or activity of the gene may affect schizophrenia.

40. The method of claim 39, wherein the compound is contacted with a product of the gene then the activity of the gene product is measured.

41. The method of claim 40, wherein the compound is contacted with the product of the gene in vitro.

42. The method of claim 40, wherein the compound is contacted with a cell that expresses the product of the gene such that the compound contacts the product of the gene.

43. The method of claim 39, wherein the compound is contacted with a cell that is capable of expressing the gene, and expression of the gene is measured and compared to expression of the gene in a cell that is not contacted with the compound.

44. The method of claim 39, wherein the compound is administered to a mammal and activity of a product of the gene is measured and compared to activity of the product of the gene in a mammal that is not administered the compound.

45. The method of claim 39, wherein the compound is administered to a mammal and expression of the gene is measured and compared to expression of the gene in a mammal that is not administered the compound.

46. The method of claim 39, wherein the gene is the CSF2RA gene, the RGS18 gene, the RGS1 gene, the IL1F8 gene, the LRP1B gene, the ROBO2 gene, the RUNX1 gene, the MN1 gene, the GPR116 gene, the GPR110 gene, the MTPN gene, the CHRM2 gene, the GRIK4 gene, the LRRC35 gene, the CDH8 gene, or the CDH11 gene.

47-72. (canceled)
